(19) United States
(12) Patent Application Publication
(10) Pub. No.: US 2020/0188468 A1
(43) Pub. Date: Jun. 18, 2020

(54) A COATED COSTUS COMPOSITION ENRICHED WITH TRITERPENOIDS AND A METHOD OF PREPARATION OF THE SAME

(71) Applicant: BENNY ANTONY, ANGAMALI (IN)

(72) Inventor: BENNY ANTONY, ANGAMALI (IN)

(21) Appl. No.: 16/628,711

(22) PCT Filed: Jul. 6, 2018
(86) PCT No.: PCT/IB2018/055011

§ 371 Date: Jan. 6, 2020

(30) Foreign Application Priority Data
Jul. 7, 2017 (IN) 201741023879

(51) Int. Cl. A61K 36/88; A61K 9/50; A61K 9/00

(52) U.S. Cl. CPC A61K 36/88; A61K 9/0053; A61K 9/5047

(57) ABSTRACT

The present invention relates to an enteric coated Costus composition with anti-diabetic activity which is enriched with triterpenoids and is made into a dosage form for the treatment of Type-1 and Type-2 diabetes. The Costus Composition is derived from Costus extract derived from Costus plant part. A process of solvent extraction and purification of said extract is also provided. A method for targeted delivery of Costus composition into intestine is provided. A dosage form is provided for the Costus composition. The enrichment with triterpenoids is up to 95% of the composition. Costus composition can lose its activity in acidic condition of the stomach. Hence by giving enteric coating to Costus Composition, it can withstand the acidic condition of the stomach thereby enhancing the bioavailability and bioactivity of the composition.

### FIELD OF INVENTION

The present disclosure relates to a coated costus composition with anti-diabetic activity and more specifically said composition enriched with triterpenoids from costus extract which is made into a dosage form for the treatment of Diabetics. A method of preparing of the said medicinal composition also is disclosed here.

### BACKGROUND OF THE INVENTION

Chronic hyperglycemia is associated with diabetes, dysfunction and failure of various organs, especially the eyes, kidneys, nerves, heart, and blood vessels. Hyperglycemia can be managed with a controlled diet, but if the conditions still prevail, it most likely leads to diabetes. Diabetes is when the body does not produce enough insulin or cells are resistant to the effects of insulin produced by the body and if the blood sugar levels stay elevated for long periods of time, and if left unchecked it can cause long-term complications.

Globally, the number of people with diabetes is expected to rise from the current estimate of 150 million to 220 million in 2010 and 300 million in 2025. The prevalence diabetes is increasing in the developing countries such as India, other south east nations. The estimated number of diabetes patients in India was around 19.4 million in 1995 and is expected to be 57.2 million in 2025 (W.H.O). In the United States, it is estimated that as of 2011, 25.8 million people (8.3% of the total population) were diabetic. According to American diabetes association about 208,000 of people under the age of 20 are estimated to have diagnosed diabetes (0.26% of all people in this age group). Approximately one in every 400-500 children and adolescents has Type-1 diabetes. In the age group of 20 years or older, 25.6 million (11.8% in men and 10.8% in women) have diabetes. In people above the age of 60 years, 10.9 million (26.9% of all people in this age group) have diabetes.

There are basically two types of diabetes Type I and Type II. Type-I diabetes, which was previously called insulin dependent diabetes mellitus (IDDM) or juvenile-onset diabetes. This develops when the body's immune system destroys pancreatic beta cells (β-cells), which are the only cells in the body that make the hormone insulin that regulates blood glucose level. Type-II diabetes was previously called non-insulin dependent diabetes mellitus (NIDDM) or adult onset diabetes. It usually begins as insulin resistance, a disorder in which the cells of the body fails to respond to insulin properly.

Anti-diabetic herbal formulations are marketed now in plenty, most of which are not subjected to standard clinical trials. It is doubtful whether these preparations are effective and there are any side effects. A limiting factor with herbal products is the lack of consistency in composition. Unlike modern medicines, herbal blends and compositions derived from plants are not standardised for pharmaceutical use.

Costus is a medicinal plant native to Mexico. This plant with a distinct yellow flower is distributed along the coast from Mexico to Costa Rica and is locally known as Cana Agria or Cana De Jabali in Mexico. It was recently introduced to peninsular part of India where the climatic conditions are favourable for their growth. In recent times costus has gained popularity for its medicinal property. Costus commonly known as spiral ginger or painted spring ginger is from the Costaceae family, which happens to be a sub-family of Zingiberaceae. It is a perennial herb growing up to 2-3 m and spreads 1.5-2 m; it has a rhizome, leafy aerial shoot and aerial adventitious buds, which act as potential regenerators. It has long narrow leaves with characteristic wavy edges and the leaves are less fleshy and have an acrid taste. Painted spiral ginger can be recognised by its yellow flowers with red spots and stripes. Propagation is carried out through stem cuttings and rhizome.

Preliminary phytochemical evaluation of costus revealed that the leaves contain about 21.2% fibres. Successive extractions showed a presence of carbohydrates, phytosterols, saponin glycosides, flavonoids and tannins in a detectable range. A study conducted by Annadurai et al has found Bixin in the costus leaves (Next generation sequencing and de novo transcriptome analysis of costus, a non-model plant with potent anti-diabetic properties). Their finding suggests the costus can be an alternative source for bixin. It is to be noted that not every constituent is useful in a plant that includes costus.

There have been reports on the costusleaves work against cancer (Nadumane et al, Evaluation of the anticancer potential of costus pictus on fibrosarcoma (HT-1080) cell line; Journal of Natural Pharmaceuticals. April 2011, Vol. 2 Issue 2, p 72-76. 5p. 2 Colour Photographs, 5 Graphs). The leaves are also suggested to act as anti-bacterial and anti-glycation agents. Costus is also known to be a powerful diuretic agent, making it useful for the treatment of renal disorders. Costus extract (alcoholic, aqueous and juice extracts of leaves/whole plant) is reported to have anti-diabetic property (U.S. Pat. No. 7,255,886B2, Antony (2007); Nandha kumar Jothivel et al (2007); M A Jayasri et al (2008)). US patent U.S. Pat. No. 8,197,864B2; Antony (2012) has shown optimum results for solvent extracts of costus against diabetes. The study conducted by Jothiv et al (Anti-diabetic Activity of costus in Methanol Leaf Extract of Alloxan-induced Diabetic Rat) observed extract of costus pictus D. Don possesses significant anti-diabetic effects in alloxan-induced diabetic rats.

Costus is reported to contain oxalic acid (Camargo et al 0.2006; Moron et al. 2007). The leaves of costus are sour in taste due to the presence of high levels of oxalic acid in the leaves. (Rajendran Sathishraj et al, 2011).

Costus extracts have a high concentration of oxalic acid. If it is consumed in small quantity it may not be harmful and be beneficial against diabetes (Oxalic acid—Induced Modification of Postglycation Activity of Lysozyme and its Glycoforms (Hong Ying Gao et. al, 2010), but Oxalic acid is known to produce Kidney stones. (Effect of dietary oxalate and calcium on urinary oxalate and risk of formation of calcium oxalate kidney stones, Linda K Massey, 1993).

Manjula et al. have detected the presence of heat sensitive proteins and total soluble proteins in costus and found to have antimicrobial properties (Valuation of Heat Stable Proteins, Total soluble Proteins and Antibacterial Properties of costus Pictus D. Don). It is not yet found out if the proteins do have any anti-diabetic property or can it be used as an alternative to insulin. More work is needed towards purification of protein and standardises it for pharmacological use. Multiple medical/health benefits are associated with costus. These benefits are the direct or indirect actions of the multiple phytochemicals present in costus. Identifying individual phytochemicals and mapping their pharmacological activity will enhance the efficacy of the final product and eventually reduce the cost of production. Moreover, it is also important to identify the components present in costus which are responsible for the anti-diabetic activity. Stability and absorption of these identified phytochemicals have to be determined for better delivery and bioavailability.

The primary work done towards costus is directed towards its leaf extract and in particular, methanol extracts. Traditionally the methanol extract of costus is attributed with anti-diabetic property and in comparison to ethyl acetate and hexane, methanol gives a higher yield. But not all the molecules and polyphenol that get extracted with methanol have any activity. For better efficacy, the active components must be identified and a commercially feasible process of extraction should be developed.

Previous work from the inventor has shown anti-diabetic activity for various Costus pictus D. Don extracts (U.S. Pat. Nos. 7,378,113, 7,635,495, 7,939,114, 8,197,864, 8,663,713), but it fails to identify a specific constituent from extracts which provide such activity more over there is a limitation the costus constituents have which was not identified in the previous work. The inventor has conducted series of in vitro studies and clinical trials on costus and it was observed that the costus loses much of its anti-diabetic activity at lower pH. The inventor dedicated that the pharmacodynamics of Costus can be improved significantly if it is not exposed to the acidic environment of the stomach.

### OBJECTIVES OF THE INVENTION

One aspect is to preserve the bioactivity of costus constituents and enhancing the pharmacodynamics properties of said constituents. The disclosure provides a composition to overcome the limitation of costus at lower pH, and a process to manufacture said composition.

Another object is to increase the bioavailability of costus constituents for the treatment of diabetes and hyperdyslipidemia. Bioavailability is enhanced by targeted delivery of the constituents to achieve the desired pharmacodynamics.

Another objective is to provide a method to segregate and enrich constituents from costus extract which have clinically significant effect in the treatment of diabetic and hyperlipidemia. Compositions with said enriched constituents are also provided.

### SUMMARY OF THE INVENTION

Disclosure provides an enteric coated composition having a core and an enteric coating layer. The core has a Costus pictus constituent and a pharmaceutical carrier. The Costus pictus constituent and the pharmaceutical carrier are blended in a ratio of about 10:1 to about 1:10.

The Costus pictus constituent is selected from the group consisting of Costus pictus plant juice, concentrated Costus pictus plant juice, powder of dried leaf of Costus pictus, solvent extract of Costus pictus, polyphenol enriched extract of Costus pictus, protein enriched extract of Costus pictus, water insoluble triterpenoid enriched extract of Costus pictus and combinations thereof.

In the enteric coated composition, the Costus pictus constituent includes water insoluble extract of Costus pictus leaf which is enriched with triterpenoids. The Costus pictus composition comprises by weight: about 10% to about 95% triterpenoids and about 0.1 to about 1% oxalic acid. The triterpenoids comprises by weight about 15% to 80%. The triterpenoids comprises by weight about 35% to 50%. The enteric coated composition of claim 3, wherein the triterpenoids comprises by weight about 80% to 90%.

The pharmaceutical carrier is selected from the group consisting of polyvinyl-pyrrolidone, cellulose derivatives including hydroxypropyl methylcellulose and hydroxypropyl cellulose, cyclodextrins, gelatines, hypromellose phthalate, sugars and polyhydric alcohols, Calcium Sulphate Dihydrate, Dibasic Calcium Phosphate, Sorbitol, Mannitol, Anhydrous Lactose, Dextrose, gums, Gum Acacia, Gum Tragacanth, Ethylcellulose, Methylcellulose, Polyvinyl alcohol, sodium carboxy methyl cellulose, silicon dioxide, polyethylene glycol 400, Polyethylene glycol 4000, starch, porous silica carriers, aluminum silicate, calcium silicate, sugars, magnesium stearate, cellulose, calcium phosphate and combinations thereof. The pharmaceutical carrier is a modified starch.

The enteric coating material is selected from the group consisting of combination of aqueous ethyl cellulose and sodium alginate, poly (methacrylic acid-co-methyl methacrylate), cellulose acetate phthalate, cellulose acetate trimellitate, hydroxypropyl methylcellulose phthalate, hydroxypropyl methylcellulose acetate succinate, and combinations thereof.

The enteric coating material is a combination of aqueous ethyl cellulose and sodium alginate. The enteric coating layer further comprises an excipient selected from the group consisting of ethyl cellulose, sodium alginate, esters of aleurtic acid poly (vinyl acetate phthalate), hydroxypropyl methylcellulose, acetaldehyde dimethyl cellulose acetate, shellac, chitosan, fatty acids, zein, waxes, plant fibers, modified starch, aluminum silicate, calcium silicate, sugars, magnesium stearate, cellulose and calcium phosphate, polysorbate, surfactants and combinations thereof.

The core is in a liquisolid form. The oral dosage form is selected from the group consisting of tablet, mini tablet, micro encapsulate, pill, capsule, soft gel capsule, and, hard gel capsule.

A method of enhancing antidiabetic activity comprising administering the enteric coated extract of Costus pictus. The antidiabetic activity is selected from the group consisting of controlling level of glucose in blood, causing regeneration of β-cells, preventing production of glucose in the liver, lowering hyperglycemia, increasing insulin secretion in Type1 diabetic patient, treating Type 2 diabetes by decreasing HbAlc level, balancing lipid profiles, increasing liver glycogen, and increasing muscle glycogen.

A method to improve yield of an extract of Costus pictus during manufacturing, the method to improve yield by combining an extract prepared from a juice of Costus pictus with an extract prepared from a pulp of Costus pictus, the method to improve yield has the flowing steps; a) Crushing plant parts of Costus pictus to extrude the juice of Costus pictus and the pulp of Costus pictus. b) Separating the juice of Costus pictus from the pulp of Costus pictus. c) Extracting the juice of Costus pictus from step b) by a method of; c1) Treating the juice of Costus pictus with ethyl acetate to obtain a liquid phase ethyl acetate extract of juice of Costus pictus; c2). Washing the liquid phase ethyl acetate extract of juice of Costus pictus of step c1) with deionized water in presence of NaCl and collecting a first ethyl acetate phase, the first ethyl acetate phase is a first water insoluble triterpenoid enriched ethyl acetate extract. c3) Concentrating the first water insoluble triterpenoid enriched ethyl acetate extract from step c2) to obtain a concentrate of the first water insoluble triterpenoid enriched ethyl acetate extract of the juice of Costus pictus.

Extracting the pulp of Costus pictus of step b) by a method of: d1) Extracting the pulp of Costus pictus extract with ethyl acetate to obtain a first liquid phase ethyl acetate extract of pulp of Costus pictus. d2) Washing the liquid phase ethyl acetate extract of pulp of Costus pictus of step d1) with deionized water and NaCl and collecting a second ethyl acetate phase, wherein the second ethyl acetate phase is a second water insoluble triterpenoid enriched ethyl acetate extract of the pulp of Costus pictus; d3) Concentrating the second water insoluble triterpenoid enriched ethyl acetate extract of the pulp of Costus pictus from step d2) to obtain a concentrate of the second water insoluble triterpenoid enriched ethyl acetate extract of the pulp of Costus pictus.

Combining the concentrate of the first water insoluble triterpenoid enriched ethyl acetate extract of juice of Costus pictus of step c3) with the concentrate of the second water insoluble triterpenoid enriched ethyl acetate extract of the pulp of Costus pictus of step d3) to obtain a third water insoluble triterpenoid enriched extract of Costus pictus, wherein separately extracting then combining extracts prepared from the juice of Costus pictus and the pulp of Costus pictus results in an improvement in yield of extract from Costus pictus.

A method of enriching triterpenoids by the following steps: Dissolving the improved yield extract of Costus pictus in methanol and separating a solvent phase and a residue. Loading a wet packed column having HP20 in methanol with the solvent phase. Eluting the product of step h) with ethyl acetate to obtain an ethyl acetate eluate and, concentrating the ethyl acetate eluate to obtain a triterpenoid enriched extract of Costus pictus. The triperpenoid enriched extract of Costus pictus comprises about 80% to about 90% w/w of triterpenoids.

A method of preparing an enteric coated composition with a core and an enteric coating layer, the core is made of a Costus pictus constituent and a pharmaceutical carrier, and, the enteric coating layer comprises an enteric coating material,

The method includes, mixing a Costus pictus constituent and pharmaceutical carrier to obtain a first uniform blend. Spray drying the uniform blend to obtain a powder. Fluidizing the powder. Spraying a coating solution onto the fluidized powder to obtain granules of the enteric coated composition comprising a core and an enteric coating layer, wherein the core comprises the Costus pictus constituent and the pharmaceutical carrier, and, wherein the Costus pictus constituent and the pharmaceutical carrier are blended in a ratio of 10:1 to 1:10.

### BRIEF DESCRIPTION OF THE DRAWINGS

Various features and advantages of the subject matter should become apparent from the following figures of the accompanying drawings, which demonstrate the process steps involved in various process trials carried under certain embodiments as well as the findings.

FIG. 1. Process to extract costus plant parts using Methanol as solvent.

FIG. 2. Process to prepare a two stage Ethyl acetate extract with costus.

FIG. 3. Process to enrich Triterpinoids from Ethyl acetate extract of costus.

FIG. 4. Method to prepare a coated granule of costus.

### DETAILED DESCRIPTION OF THE INVENTION

A costus composition having an active core and a layer of coating over the core. The active core is made from constituents derived from costus. The coating facilitates targeted delivery of costus constituents in any part of gastrointestinal system. A method of preparing said composition is also disclosed. Further art and scope related to several aspects of work also are disclosed here, which includes an understanding of problem solved, their background and conventional technologies.

Costus includes; costus igneus Nak, Costus pictus D. Don (Costus pictus), costus Mexicans Liebm ex Petersen or, commonly known as fiery costus, step ladder or spiral flag or insulin plant. They are native to South and Central America, the plant species have been recently introduced to India and is mostly grown as an ornamental plant in the tropical regions of India.

The Active core includes of costus constituents. The active core is in a liquisolid powder or granule form. The costus constituents are selected from a group consisting of costus plant juice, leaf juice, a concentrate of concentrated Costus pictus plant juice, costus dried plant part or extract of costus plant parts, more specifically polyphenols, protein, or triterpenoids derived from costus and a combination of thereof. The plant part can include root, shoot, leaves, flower and rhizome, or leaf.

The Liquisolid technique, which is based on the conversion of the drug in liquid state into an apparently dry, non-adherent, free flowing and compressible powder. Throughout the specification the liquisolid system is also referred to as ‘liquisoid form’ or ‘liquisolid’ per se.

It was observed that at low pH, the activity (towards diabetes) of costus extract is lost or declines significantly. In vitro study on the stability of costus extract in gastrointestinal conditions was conducted. It was found that, for better activity the extract of costus should be protected from acidic environment, such as the stomach. The activity precisely to be protected is the anti-diabetic activity of the extract of costus. In vivo studies data supported the in vitro study predictions. The costus constituents that are protected from stomach acids showed better activity. The targeted delivery significantly reduced the dosage requirement.

A vivo study made it clear that targeted delivery enhances pharmacodynamic properties of costus constituents and their bioactivity. It is contemplated that, enhancement in bioactivity is a direct result of enhanced bioavailability from the intestine.

Anti-diabetic activity includes controlling the level of glucose in blood, regeneration of β-cells, preventing the production of glucose in the liver and to managing hyperlipidemia. Treatment for Diabetes with respect to present disclosure includes Type1 and Type 2 diabetes.

Anti-diabetic activity also includes decreasing HbAlc level, balance lipid profile, increase liver and muscle glycogen and increasing insulin secretion in a diabetic or hyperglycaemic patient. The benefits are not strictly bound to diabetes, one skilled in the art can deduct benefits in other aspects of human health which may be directly or indirectly associated with diabetes, such as kidney function, fatigue, nausea, and so on.

The dose form, when necessary, is selected from a group consisting of a capsule, tablet, granule, powder sachet, pill, sustained release formulation, paste, ointment, infusion, injection, ampoule, solution, suspension, emulsion, and combinations thereof.

In some embodiments pharmaceutically acceptable excipients are used, they are selected but not limited to of polyvinyl-pyrrolidone, cellulose derivatives including hydroxypropyl methylcellulose and hydroxypropyl cellulose, cyclodextrins, gelatines, hypromellose phthalate, sugars and polyhydric alcohols, Calcium Sulphate Dihydrate, Dibasic Calcium Phosphate, Sorbitol, Mannitol, Anhydrous Lactose, Dextrose, gums, Gum Acacia, Gum Tragacanth, Ethylcellulose, Methylcellulose, Polyvinyl alcohol, sodium carboxy methyl cellulose, silicon dioxide, polyethylene glycol 400, Polyethylene glycol 4000. The composition may also have suitable additives for pharmaceutical use such as preservatives, stabilizers, surface active agents, emulsifiers, salts for the regulation of the osmotic pressure, buffers, flavouring agents and colouring agents.

Costus has anti-diabetic activity, the activity can be enhanced. The disclosure provides a costus composition with enhanced anti-diabetic activity in comparison to costus plant parts or concentrates or extract. The costus composition has an active core and a layer of coating over the core. The core is primarily made up of costus constituents and some pharmaceutical carrier. The pharmaceutical carrier can be an absorbent, any form of starch, porous silica carriers, aluminum silicate, calcium silicate, sugars, magnesium stearate, cellulose and calcium phosphate, binder, surfactant, filler or a film that encapsulated the Costus constituents.

The coating facilitates targeted delivery of costus constituents. According to the desired delivery site (the gastrointestinal system) for the core the nature of the coating material changes. In the present disclosure it is desirable to protect the core from the stomach acids, so the coating protects the core from stomach acid and release the core after leaving the stomach. The costus composition can release the core in duodenum, jejunum, ileum, cecum, colic flexures, transverse mesocolon or colon, but preferably in duodenum.

In some embodiment of costus composition, the coating may facilitate sustain released of constituents from the core, where the constituents from the core will be released in a steady manner for a set duration of time.

In one embodiment of costus composition, the core is provided with a coating which can sustain the stomach acid but start dissolving as the pH reaches 5. The costus composition releases the core in small intestine, more precisely at duodenum.

In one embodiment of costus composition, the thickness of the coating material determines the time of delivery, the coating over the core is provided enough to release the core at the small intestine, preferably duodenum. The coating material used is a polymer that dissolves in the gastric juice with time.

In another embodiment of costus composition, the coating material is selected from the group consisting of poly (methacrylic acid-co-methyl methacrylate), esters of aleurtic acid, cellulose acetate phthalate, cellulose acetate trimellitate, poly (vinyl acetate phthalate), hydroxypropyl methylcellulose, hydroxypropyl methylcellulose phthalate, hydroxypropyl methylcellulose acetate succinate, acetaldehyde dimethyl cellulose acetate, sodium alginate, ethyl cellulose, chitosan, zein, fatty acids, waxes, shellac, plastics, plant fibers, and, a combination of ethyl cellulose and sodium alginate. The basic property of coating material is such that, it retains its structure in the stomach but dissolves and releases the active ingredients (primarily costus constituents) as soon as the composition passes the stomach.

In one embodiment, main constituent of the core of the costus composition is a costus extract. Disclosure provides costus extract enriched with triterpenoids for the costus composition. The costus extract is enriched with triterpernoids by weight from about 10% to about 95%, or from about 15% to about 80%, or about 35% to about 50%. An extract of costus is provided with a purity of triterpenoids by weight up to 90%. Some embodiments provide triterpenoids by weight from about 80% to about 90%.

One aspect provides a triterpenoids enriched costus extract. Some embodiments provide a powdered formulation made with enriched costus extract. The powdered formulation has a triterpenoids purity by weight of about 75%, or about 30% to about 60%. It is further noted that enrichment of Triterpenoids has given an improved efficacy to costus extract, that is, a significantly lower dose is required for the same effect as a general solvent extract per se.

It is further noted that in some embodiments is a powdered costus formulation, wherein purified costus extract, a liquid, is absorbed onto suitable absorbent. The suitable absorbent and/or binders are selected from a group but not limited to starch as Calcium Sulphate Dihydrate, Dibasic Calcium Phosphate (DCP), Sorbitol, Mannitol, Anhydrous Lactose, Dextrose, Gum Acacia, Gum Tragacanth, Synthetic Polymeric binders Ethylcellulose, Methylcellulose, PVA, Sod CMC, Silicon Dioxide, PEG 400, PEG 4000 or a combination thereof.

The binders and absorbents are not limited by the few disclosed above, one skilled in the art can use any combination of the above-given absorbents and binders or opt for other suitable binders and absorbents for making powder, granule, tablets from costus extract or constituents.

In one embodiment the Triterpenoids enriched costus extract composition is provided in a dosage ranging from 10 mg to 1000 mg, or about 100 mg to about 500 mg, or about 150 mg to about 350 mg, or about 200 mg to about 300 mg. The dosages are provided in dose forms selected from; Capsule, tablet, granule, sachet, pill, micro encapsulates, sustained release formulation, paste, ointment, infusion, injection, ampoule, solution, suspension, emulsion, and combinations thereof.

In one embodiment costus Composition made with a triterpenoids enriched powdered formulation of costus extract. The costus extract with a purity of about 20% to 95% triterpenoids by weight, or about 25% to about 70% triterpenoids by weight, or about 30% to 60% triterpenoids by weight. Triterpenoids includes terpenoids which are the hydrocarbons of plant origin of the general formula (C5H8) n as well as their oxygenated, hydrogenated and dehydrogenated derivatives.

Yet another aspect provides a method of coating the costus constituents by the following steps. In an agitating vessel costus extract is taken, HI-CAP® (Ingredion India Pvt Ltd, Mumbai, India), a modified food starch derived from waxy maize especially suited for the encapsulation of flavours, clouds, vitamins and spices, at high oil loading. It is characterised by excellent resistance to oxidation. This product is recommended as a total replacement for expensive encapsulating agents such as gum Arabic and gelatine. It can also be used to encapsulate other water insoluble liquid or solid substances such as vitamins and fatty esters. Polysorbate is added optionally into the agitator and mixed to form a uniform blend. The mixture is spray dried to make it in powder form. The specific quantity of powder is loaded into the bowl of the fluid bed extractor (the bowl has a fine Stainless steel mesh at the bottom). Hot, filtered air up to 90° C. was passed at high velocity from the bottom of the FBE bowl through the feed material and feed material was fluidised (The air used for drying/fluidising was successively filtered). Meantime coating material, let's say (Poly-methacrylicacid-co-methyl) methacrylate (Eudragit), is dissolved in water. The coating solution was sprayed into a fluidised material by using a spraying device attached to the FBE. Through the process of fluid bed coating, fluidised particles are continuously sprayed with the coating solution, depositing layers (films) of coating material onto the surface of the particles, and yielding an even layer, 2% to 7% weight gain with an area density of 2 to 8 mg/cm² thickness.

Another embodiment provides a dosage for costus composition for oral use, wherein the dosage ranges from 10 mg to 1000 mg, or about 100 mg to about 500 mg, or about 150 mg to about 350 mg, or about 200 mg to about 300 mg per day for an average diabetic human being. In a dosage, 80%-40% of the composition is some pharmaceutically acceptable excipient.

In another embodiment the costus composition dosages are provided in dose forms selected from; capsule, tablet, granule, powder, sachet, pill, sustained release formulation, paste, ointment, infusion, injection, ampoule, solution, suspension, emulsion, and combinations thereof. Preferably but not necessary, the costus composition is blended with a suitable pharmaceutical excipient before making into dose form. The final costus composition form will have about 80%-40% of excipient.

In yet another embodiment, the costus extract with a purity of about 20% to 95% by weight of triterpenoids, or about 25% to 70% triterpenoids, or about 30% to 60% triterpenoids is blended with pharmaceutical carrier in a ratio of about 10:1 to about 1:10, or about 1:4 to about 3:2.

Another aspect is a process to enhance the pharmacodynamic activity of costus constituents, especially triterpenoids towards diabetes by delivering costus constituents in the form of the costus composition. That is, by protecting the costus constituents from the stomach acid and delivering it directly at the intestine, preferably at duodenum.

In one embodiment a method to treating diabetes is provided, by administering a patient with the costus composition. The costus composition has anti-diabetic activity. The costus composition can be used for the treatment of Type1 or Type 2 diabetes or can be used along with other drugs used for treatment of diabetes. The costus composition can also be made part of a formulation for the treatment of diabetes along with other active ingredients.

In one embodiment, a method to preserve the bioactivity of costus constituents is provided and the bioactivity is directed towards Diabetes. Method includes stepl; a solvent extract of costus plant part is blended with excipients to make it in a dry powder form, Step 2; the dry powder extract is made into granules, for example, by adding some edible adhesive, step 3; the granules are provided with a layer of coating (using fluidised bed), Step 4; final granules with coating are dried. The coated costus extract is the costus composition with enhanced bioactivity. For the enhanced bioactivity, the coating material protects the costus constituents from digestive enzymes and gastric acid of the stomach.

In one embodiment costus composition is used for the treatment of dyslipidemia, more particularly to lower total cholesterol (TG), Low-density lipoprotein (LDL), very low density lipoprotein (VLDL) and triglyceride in a patient in need thereof. Regular intake of the costus composition with the coating in the range of 1 mg/kg to 25 mg/kg body weight of a patient will maintain blood sugar level and also balance lipid profile within 4 weeks.

In another embodiment, a method is provided to control the blood glucose level on a carbohydrate rich diet, by blending costus composition with carbohydrate rich food product. Carbohydrate rich food includes but not limited to, sweet candy, pastries, cakes, bread spreads, fruit juice, fruit jams, peanut butter, pie, pudding, mayonnaise, jellies and thereon.

In some embodiment the core of the costus composition has other active constituents along with costus constituents. The core may also include active constituents which are derived from other plants.

In one embodiment costus constituents can also be blended with polyphenols and antioxidants derived from other plants to make the core of the costus composition. Other plants are selected from but not limited to Piper longum Linn, Terminalia chebula Retz, Murraya koeniggii, Ginger, Glycyrrhia glabra, turmeric, Centella asiatica, Cyperus rotundus and Piper nigrum.

Yet another is a process to extract high yield extract of costus with less than 1% oxalic acid. The process involves the following steps. Crushing the plant parts, followed by separating the juice and pulp, then extracting both the parts separately with suitable solvents. Next combine the extracts of juice and pulp, and wash the extract with water. Thus finally high yield extract with less than 1% oxalic acid is obtained. The solvents for extraction can be selected from but not limited to water, hexane, methanol, ethanol, isopropanol, n-butanol, methyl acetate, ethyl acetate, propyl acetate, n-butyl acetate and combinations thereof to obtain an extract. In some embodiments the solvent is Ethyl acetate. The plant parts used for solvent extraction include; stem, leaf, rhizome and combination thereof. The solvent extract of costus plant is further purified using chromatography to enrich triterpenoids extract of costus.

In another embodiment, a method to extract high yield costus extract is provided, the process includes the following steps. Plant parts of costus including the leaf, stem, and rhizome are washed to remove any foreign material. Then the plant parts are crushed into a pulp form. Juice is separated from the pulp. The process requires extracting the juice and pulp separately with Ethyl acetate to enhance the yield and purity of extract during the manufacturing process. The pulp of costus is treated with Ethyl acetate, the mix is stirred well and left aside, the Ethyl acetate part is separated. The juice of costus is treated with an equal quantity of Ethyl acetate, the mix is stirred well and left aside, two layers are formed and the Ethyl acetate part is separated. The concentrated Ethyl acetate extract of costus pulp and juice is washed with water so as to remove water soluble impurities including Oxalic acid from the extract. NaCl is also added in the water washing process. The Ethyl acetate extract of costus pulp and juice is concentrated under vacuum at a steady temperature. Ethyl acetate extract thus obtained has the following characteristics; (a) Oxalic acid is dropped from over 50% to less than 1%, (b) the extract obtained is water insoluble, and (c) Ethyl acetate extract of costus has a triterpenoids purity of 12%-30%, more accurately close to 30%.

In another embodiment Ethyl acetate extract of costus has a triterpenoids purity of 12%-30% is further purified to enrich triterpenoids. To start with, the ethyl acetate extract is loaded into a column. The loaded extract is eluted with pure methanol. Polyphenols, flavonoids and alkaloids get extracted out with methanol. Said column bed is first eluted with methanol five times. Then once again the column is eluted one to two times more to get Ethyl extract with enriched triterpenoids. Triterpenoids in the methanol part are about 11% and triterpenoids in Ethyl acetate is 45-70%. It is found that using flash chromatography a higher purity of 95% is achieved. Methanol part can be again recycled after the solvent is removed and recovered.

The solvents used for various extracts including column extraction are selected from but not limited to organic, polar, non-polar, alcohol, Methanol, Ethanol, Hexane, Ethyl acetate, ionic liquids, Acetonitrile, Ether, water and combination thereof. A person skilled in the art can achieve the same results with different solvents through standard experimentation.

One of the constituents of costus plant is Oxalic acid, which is responsible for the precipitation of solid calcium oxalate in Kidney when ingested at higher dose regularly. The disclosure provides a costus extract product with extremely low oxalic acid content compared to the natural counterpart and a standard solvent extract.

The following examples are for illustration and do not limit the scope of the disclosure in any way.

### Example 1

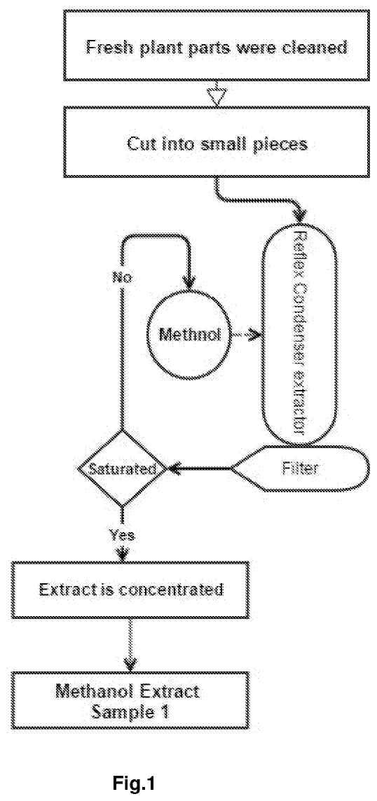

Extracting Costus with Methanol, Reference is Made to FIG. 1 Showing the Process to Extract Costus Plant Parts Using Methanol.

The fresh plant parts of costus was collected (5000 Kg) as raw material. The fresh plant parts were cleaned and cut into small pieces. About four times the quantity of raw material 90%-methanol was added to the raw material for extraction. The extraction was performed in an extractor with a reflux condenser. The bottom of the extractor was fitted with a polypropylene (100 microns) filter cloth. The mixture was refluxed at the boiling temperature (60-70° C.) of methanol. To check saturation of solvent, sample was taken for the extractor at regular intervals and tested for total-dissolved-solids (TDS), extraction was continued till solvent was saturated. The residue and supernatants were separated by draining out the supernatant from the extractor bottom through the polypropylene filter cloth using a centrifugal pump. The first residue was then further extracted; step 3 to 8 was repeated two more times. All the supernatants were pooled and concentrated in an Agitated thin film evaporator (ATFE) to form a concentrated methanol extract. The methanol extract was concentrated under vacuum at above 500 mm of mercury (66.67 KPa) to get a liquid (90% methanol) extract of fresh plant parts of costus (Sample-1), here onwards referred to as Extract-1(Ex-1) (Yield 125 Kg).

### Example 2

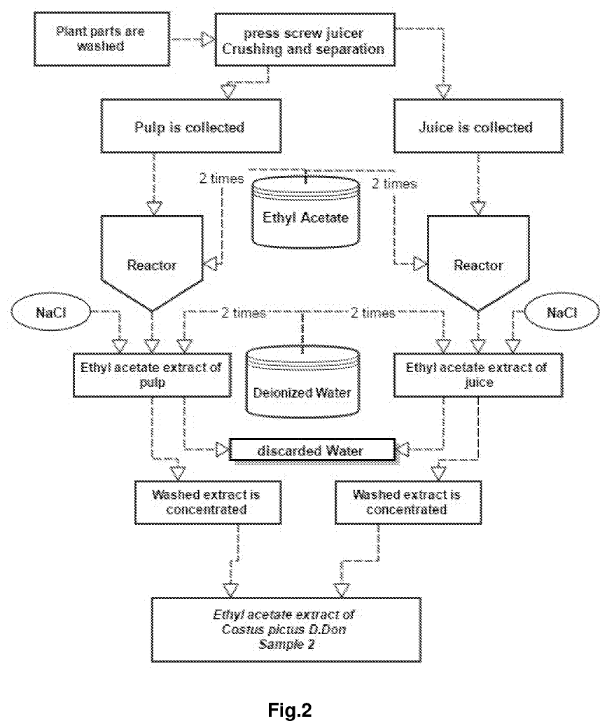

High yield extraction of costus plant part with Ethyl Acetate, a reference is made to FIG. 2 showing a process to prepare a two stage Ethyl acetate extract with costus). About 890 Kg costus areal plant parts were washed and sorted out as raw material for extraction.

About 890 Kg costus areal plant parts were washed and sorted out as raw material for extraction. The plant parts were crushed using press screw juicer. Juice and the pulp were collected in separate vessels. The Juice and the pulp were extracted separately, i.e. 172 Kg pulp extracted separately and 260 L of juice extracted separately.

Extraction of costus pulp, the 172 kg of pulp was fed into a reactor. The reactor was charged with 250 litres of Ethyl acetate. The extraction was conducted at a constant temperature of 50° C. for 30 min. The solvent was drained out and collected. The extraction is repeated three more times with fresh 250 litres Ethyl acetate. All the four drained out solvents were combined and concentrated to 350 litres at 55° C. under vacuum. The concentrated ethyl acetate extract obtained was washed with 350 litres deionised water in presence of about 7 kg of NaCl. The water and ethyl acetate phase was separated and water was drained out of the vessel. The ethyl acetate phase was washed with water in presence of NaCl two more times. Ethyl acetate phase was collected and concentrated in an Agitated thin film evaporator (ATFE) to form concentrated ethyl acetate extract (water insoluble). The concentrated ethyl acetate extract was fed into vacuum stripper and dried under vacuum at above 50° C. and 6.6 KPa. The Ethyl acetate extract of costus was concentrated till it reaches the TDS reaches 12% (Ex1).

Extraction of costus Juice, costus 260 litre juice was treated with equal volume of ethyl acetate, wherein the two layers were separated eventually. Ethyl acetate was separated from the juice, and the juice was again treated with ethyl acetate one more time. Both the ethyl acetate parts were combined and concentrated. The concentrated ethyl acetate extract obtained was washed with 350 litres deionised water in presence of about 7 kg of NaCl. The water and ethyl acetate phase was separated and water was drained out of the vessel. The ethyl acetate phase was washed with water in presence of NaCl two more times. Ethyl acetate phase was collected and concentrated in an Agitated thin film evaporator (ATFE) to form concentrated ethyl acetate extract (water insoluble). The concentrated ethyl acetate extract was fed into vacuum stripper and dried under vacuum at above 50° C. and 6.6 KPa. The Ethyl acetate extract of costus was concentrated

Ethyl acetate extract was obtained from Pulp (Ex1) and Juice(Ex2) were combined to get the high yield ethyl acetate extract of costus (Sample-2).

The HPLC analysis of the combined extract from step 8 was conducted, the triterpenoids content of the Ethyl acetate extract of costus was found out to be 20±4%.

### Example 3

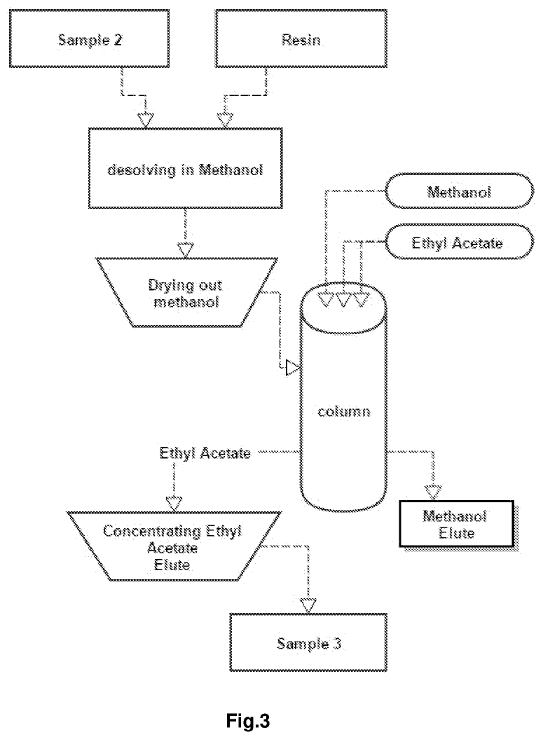

Purification of Sample 2 to Enrich Triterpenoids Using Column Chromatography-Reference is made to the FIG. 3 showing a process to enrich triterpenoids from ethyl acetate extract of costus. The Ethyl acetate extract of costus (Sample 2) was further purified to enrich triterpenoids. Sample 2 derived from the process as shown in example 2 was selected for this illustration although other costus extracts can also be enriched by the method illustrated here. The 5 kg Sample 2 was dissolved in methanol (25 TDS) and the undissolved residue was removed by centrifuge. A wet packed column was prepared with HP20 in methanol. The sample-2 dissolved in methanol from step 1 was loaded on to the column. The column was eluted with absolute methanol (a 5 beds elusion). The triterpenoids percentage in methanol elute was found out to be about 11%. The column was then eluted with Ethyl acetate (2 beds). The Ethyl acetate elutes were collected and concentrated in thin film evaporator to derive Sample-3. The triterpenoids percentage in Sample-3 was about 84±5%. The final Ethyl acetate elutes was a dark green viscous liquid.

### Example 4

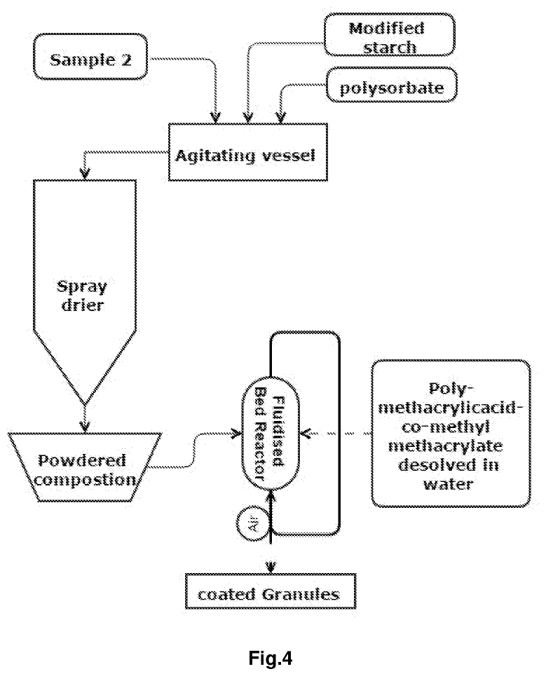

Process to Coat Costus Extract—Reference is Made to the FIG. 4 Showing a Method to Prepare a Coated Granule of Costus.

Sample-2 was chosen for this illustration, the illustration do not limit the process to only sample-2.

In an agitating vessel, 40 kg of sample 2 was taken. HI-CAP, (Ingredion India Pvt Ltd, Mumbai, India), a modified food starch derived from waxy maize especially suited for the encapsulation of flavours, clouds, vitamins and spices, at high oil loading. It is characterised by excellent resistance to oxidation. This product is recommended as a total replacement for expensive encapsulating agents such as gum Arabic and gelatine, about 55 kg of it was added to the agitating vessel. About 5 L of polysorbate (emulsifiers) was added into the agitator and mixed to form a uniform blend. The blend from step 3 was spray dried to make it in powder form. A specific quantity of powder from step 4 was loaded into the bowl of the fluid bed systems (for this illustration fluid bed from Pam Glatt Pharma Technologies Pvt Ltd was used, the maker of the fluid bed do not limit the scope of the disclosure). The bowl has a fine Stainless steel mesh at the bottom. The air used for drying/fluidising was successively filtered through HEPA (High-efficiency particulate air) filters (EU 13 grade, 0.3-micron rating, and 99.99% efficiency). Hot, filtered air up to 90° C. was passed at high velocity from the bottom of the FBE bowl through the feed material (powder from step 4) and feed material was fluidised.

Meantime 100 g coating solution was prepared by dissolving an ethyl cellulose modified by the manufacturer to form an aqueous ethyl cellulose and sodium alginate (Nutrateric®) (Colorcon 275 Ruth Rd, Harleysville, Pa. 19438 Tel: +1 215.256.7700 Fax: +1 215.256.7799) in 900 ml water.

Specifically the coating solution is prepared by Weigh the necessary quantity of water into the mixing vessel. Using the mixer and propeller stirrer, stir the water to form a vigorous vortex. Weigh the necessary quantity of NS Enteric® nutritional enteric component supplied as a dry powder, and add the powder to the water in a slow steady stream while maintaining a vigorous vortex. An increase in volume of the suspension and some foaming will occur initially, but will subside rapidly. Reduce mixer speed to low and continue to mix for 60-90 minutes to insure complete hydration. Gently agitate the Surelease (combination of ethyl cellulose, ammonium hydroxide, medium chain triglycerides, oleic acid) container to ensure complete dispersion of solids prior to dispensing the required quantity. Add this to the vessel and continue mixing for 10 to 15 minutes. The suspension should be continuously mixed during the coating process.

The coating solution was sprayed into the fluidized material by using a spraying device attached to the FBE (spray speed 0.5 L in 1 Hr, pump rpm range 10-12). Through the process of fluid bed coating, fluidised particles were continuously sprayed with the coating solution. Depositing layers (films) of coating material onto the surface of the fluidised particles, and yielding an even layer, about a 4% weight gain with an area density of 6 mg/cm² thickness.

The coated granules made from Sample-2 were called Product-2 (P2), in the same way, coated granules were made with Sample-3 and Sample-1, shall be called Product-3 (P3) and Product-1 (P1) respectively.

| 100 mg Product | Product 1 | Product 3 |
|---|---|---|
| Triterpenoids | 8 mg-10 mg | 30 mg-40 mg |
| Oxalic Acid | 0-0.4 mg | ≤0.01 mg |
| Pharmaceutical carrier | 55 mg-60 mg | 55 mg-60 mg |
| Enteric coating material | 2 mg-4 mg | 2 mg-4 mg |

### Example 5

Method to Prepare Different Costus Extracts Samples Subjected to Different pH.

Costus extract from example 2 was dissolved in methanol; the extract was dissolved in methanol to ensure solubility in the buffer. Four samples of the extract were taken and added to the different buffer (pH 1, 1.8, 3, 6.1 and 7.4) in 1:20 ratio of costus extract: buffer. The extract-buffer solutions were kept in an incubator for 3 hours bath at 37° C. for 3 hours. The solution was neutralised by adding sodium bicarbonate. The neutralised solution was concentrated and dried. The samples obtained were SpH-1.8, SpH-3, SpH-6.1, and SpH-7.4.

### Example 6

Activity Study of Sample-2 Treated in Different pH as Per Example 5.

Thirty adult Wistar rats weighing 200-250 gm were selected, at random. The animals were kept in standard animal house conditions, at 24±2° C., 65% relative humidity and 12 h light/dark cycle. The animals were acclimatized for a period of five days and fed with standard pellet diet and water. The animals were randomly divided into five groups having six rats in each.

Group 1—Normal healthy animals as control.

Group 2—Animals administered with SpH-1.8 at 75 mg/kg Body wt orally for 30 days.

Group 3—Animals administered with SpH-3 at 75 mg/kg Body wt orally for 30 days.

Group 4—Animals administered with of SpH-6.1 at 75 mg/kg Body wt orally for 30 days.

Group 5—Animals administered with of SpH-7.4 at 75 mg/kg Body wt orally for 30 days.

Animals were fasted for 12 hours and the fasting blood sugar level (FBS) was estimated for. Oral glucose tolerance test (OGTT) was performed following a glucose challenge of 2 g/kg by oral gavages. It was to test the ability of body cells to absorb glucose after you ingest a given amount of sugar. Fasting blood glucose was tested prior to administering glucose to animals. Blood glucose was recorded at 30 min (PPG₁), 60 min (PPG₂), 120 min (PPG₃) and 180 min (PPG₄), after the glucose challenge.

Average Fasting Blood glucose level (mg/dl) compared with Post prandial glucose level of rats.

TABLE 1

| | | | | | | Difference for the FBS | | | |
|---|---|---|---|---|---|---|---|---|---|
| | | PPG mg/dl | | | | value | | | |
| | FBS | 30 | 60 | 120 | 180 | 30 | 60 | 120 | 180 |
| Group | mg/dl | min | min | min | min | min | min | min | min |
| Gr 1 | 93 | 148 | 150 | 138 | 102 | 55 | 57 | 45 | 9 |
| Gr 2 | 92 | 144 | 149 | 133 | 99 | 52 | 57 | 41 | 7 |
| Gr 3 | 90 | 139 | 142 | 127 | 96 | 49 | 52 | 37 | 6 |
| Gr 4 | 94 | 134 | 122 | 105 | 92 | 40 | 28 | 11 | −2 |
| Gr 5 | 92 | 136 | 124 | 107 | 93 | 44 | 32 | 15 | 1 |

In Group 1, which was the control group, one can observe a slight spike in the blood sugar in first 60 minute and then a steady decrease in blood sugar level during the 3 hours study period. The pattern observed in group 1 animals was also observed in group 2 and Group 3 animals. Animals from Group 4 and Group 5 showed decrease in blood glucose much faster, under 120 min the blood glucose level came close to normal where as it took close to 180 min for animals in group 1 and 2 to reach normal level. Another significant difference observed was that there was a difference observed was a spikes in the blood glucose level within an hour of the glucose challenge test, but such spick were not observed in Gr 5 and Gr 4 animals.

The difference from the base line which was the fasting blood sugar to the blood sugar level was also observed. For group 1 to group 3 the difference was close to 50 in first 60 minutes, and there was a slight increase in difference from the base line. For group 4 and group 5 the difference was close to 40 at 30 minutes and it reduced steadily to close to 10 to 15 by 120 minutes.

By the end of 3 hours the difference from base line was close to 10 points for group 1 and group 2, the difference was slightly smaller for group 3. Within 2 hours the difference from base line was close to 10 to 15 for group 4 and group 5. Within three hours the difference from the base line for group 4 and group 5 was negligible.

It was established that SpH-6.1 and SpH-7.4 given to animals in group 4 and group 5 have positive effect on reducing blood glucose. As the only difference among the samples administered to animals was the pH they were subjected to, so it was also established pH do affect the activity of costus, especially towards reducing blood glucose level. Acidic pH, close to 3 and below hinders the costus activity.

### Example 7

Postprandial Glucose Levels Study of Costus in Various Dosages.

Adult Wistar rats weighing 200-250 gm were selected at random; the animals were kept in standard animal house conditions at 24±2° C., 65% relative humidity and 12 h light/dark cycle. The animals were acclimatized for a period of five days and fed with standard pellet diet and water. Six healthy rats were separated at random from the lot and diabetes was induced in rest by a single intra-peritoneal injection of streptozotocin (STZ) at 35 mg/kg. Fasting blood sugar (FBS) was measured after seven days of STZ injection and rats having FBS more than 200 mg/dl were considered as diabetic. The diabetic rats were separated out and randomly divided into 11 groups having six rats in each. Six normal healthy rats were allocated to Group I; STZ induced diabetic rats were randomly allocated to Groups 2 to 12.

The animals were tested for glucose tolerance test per below experimental design:

Oral glucose tolerance test (OGTT) was performed following a glucose challenge of 2 g/kg by oral gavages. It was to test the ability of body cells to absorb glucose after you ingest a given amount of sugar. Blood glucose was recorded at 30 min, 60 min, 120 min and 180 min, after the glucose challenge on day of the study.

Just after glucose challenge test a specific Doses of Sample-1 (from example 1) and Product-2 (from example 4) were administered through oral gavages. The dosages for Sample-1 and Product-1 were in a range of 25 mg/kg Body wt to 300 mg/kg Body wt. In one of the groups animals were administered with glibenclamide.

Group 1—Normal healthy animals as control.

Group 2—Positive control—diabetic animals administered with glibenclamide at 1 mg/kg Body wt orally for 30 days.

Group 3—diabetic animals administered with Sample-2 at 25 mg/kg Body wt orally for 30 days.

Group 4—diabetic animals administered with Sample-2 at 50 mg/kg Body wt orally for 30 days.

Group 5—diabetic animals administered with Sample-2 at 75 mg/kg Body wt orally for 30 days.

Group 6—diabetic animals administered with Sample-2 at 100 mg/kg Body wt orally for 30 days.

Group 7—diabetic animals administered with Sample-2 at 150 mg/kg Body wt orally for 30 days.

Group 8—diabetic animals administered with Sample-2 at 300 mg/kg Body wt orally for 30 days.

Group 9—diabetic animals administered with Product-2 at 25 mg/kg Body wt orally for 30 days.

Group 10—diabetic animals administered with Product-2 at 50 mg/kg Body wt orally for 30 days.

Group 11—diabetic animals administered with Product-2 at 75 mg/kg Body wt orally for 30 days.

Group 12—diabetic animals administered with Product-2 at 100 mg/kg Body wt orally for 30 days.

Group 13—diabetic animals administered with Product-2 at 150 mg/kg Body wt orally for 30 days.

Postprandial glucose level (mg/dl) of rats in streptozotocin-induced diabetes. The average blood glucose level of animals is provided in the table.

TABLE 2

| | | Postprandial glucose level | | | |
|---|---|---|---|---|---|
| | | (mg/dl) | | | |
| | | 30 | 60 | 120 | 180 |
| | | min | min | min | min |
| Group 1 | | 188 | 127 | 96 | 87 |
| Group 2 (1 mg/kg) | | 294 | 170 | 127 | 101 |
| Group 3 (25 mg/kg) | Sample-2 | 294 | 270 | 205 | 132 |
| Group 4 (50 mg/kg) | | 295 | 265 | 191 | 132 |
| Group 5 (75 mg/kg) | | 287 | 252 | 177 | 120 |
| Group 6 (100 mg/kg) | | 291 | 247 | 168 | 110 |
| Group 7 (150 mg/kg) | | 289 | 202 | 144 | 95 |
| Group 8 (300 mg/kg) | | 284 | 198 | 140 | 91 |
| Group 9 (25 mg/kg) | Product-2 | 287 | 243 | 120 | 103 |
| Group 10 (50 mg/kg) | | 292 | 248 | 119 | 102 |
| Group 11 (75 mg/kg) | | 294 | 249 | 111 | 91 |
| Group 12 (100 mg/kg) | | 286 | 243 | 111 | 91 |
| Group 13 (150 mg/kg) | | 288 | 244 | 109 | 89 |

Sample-2 was administered among group 1 to group 8 at dosages from 25 mg/kg body weight to 300 mg/kg body weight. It was observed that at lower dose (Up to a dosage of 50 mg/kg body weight) there was no significant reduction seen in blood glucose level. At a dose of 100 mg/kg to 150 mg/kg body weight of rat, clinically significant effects (reducing blood glucose) were seen. After 180 min the reduction in blood glucose seen in group 6, group 7 and group 8 were quite similar. That means increase in dose of sample-2 did not result in a faster reduction or efficient reduction in blood glucose.

Product-2 was administered among group 1 to group 8 at dosages from 25 mg/kg body weight to 150 mg/kg body weight. It was observed that at a lower dose of 25 mg/kg body weight of rat, there was clinically significant reduction seen in blood glucose level. At a dose of 25 mg/kg to 75 mg/kg body weight of rat, improvement in glucose reduction was seen. The improvement in blood glucose reduction for 25 mg/kg of product-2 was better than 75 mg/kg of sample-2. From the study and the data provided in table 2 it was evident that product-2 at a lower dosage has an efficacy greater than Sample-2. Even though the active ingredient in Product-2 and sample-2 were same, this improvement in efficacy was because product-2 was designed to release the active ingredient in the intestine.

### Example 8

Postprandial Glucose Levels Study of Costus in Various Dosages.

Adult Wistar rats weighing 200-250 gm were selected at random; the animals were kept in standard animal house conditions at 24±2° C., 65% relative humidity and 12 h light/dark cycle. After five days of acclimatisation, diabetes was induced by injecting streptozotocin 35 mg/kg dissolved in 0.1M citrate buffer of pH 4.5, intraperitoneally, as standardised in our laboratory. Five days after induction of diabetes (day 1 of the study), animals fasted for 12 hours and the fasting blood glucose level (FBG) was estimated for diagnosing diabetic rats. Animals having FBG more than 250 mg/dl were included in the study.

Six normal healthy rats were allocated to Group I; streptozotocin-induced diabetic rats were randomly allocated to Groups 2 to 9 with 6 rats in each group. The animals were treated for 30 days as per below experimental design:

Group 1—Normal healthy animals as the control.

Group 2—Diabetic control—untreated diabetic animals.

Group 3—Positive control—diabetic animals administered with glibenclamide at 1 mg/kg Body wt orally for 30 days.

Group 4—diabetic animals administered with Sample 1 at 100 mg/kg Body wt orally for 30 days.

Group 5—diabetic animals administered with Sample 2 at 100 mg/kg Body wt orally for 30 days.

Group 6—diabetic animals administered with of Sample 3 at 100 mg/kg Body wt orally for 30 days.

Group 7—diabetic animals administered with Product-1 at 50 mg/kg Body wt orally for 30 days.

Group 8—diabetic animals administered with Product-2 at 50 mg/kg Body wt orally for 30 days.

Group 9—diabetic animals administered with of Product-3 at 10 mg/kg Body wt orally for 30 days.

FBG was measured on Day 0, Day 15 and Day 30. OGTT was also performed on Day 0, Day 15 and Day 30.

Fasting blood glucose level (mg/dl) of rats in streptozotocin-induced diabetes. The average blood glucose level of animals in each group is provided in table 3.

TABLE 3

| | | 0^{th} | 15^{th} | 30^{th} |
|---|---|---|---|---|
| | Groups | day | day | day |
| Control | Group 1 | 91 | 92 | 88 |
| | Group 2 | 358 | 493 | 495 |
| | Group 3 | 388 | 266 | 142 |
| Extracts | Group 4 | 389 | 252 | 175 |
| | Group 5 | 387 | 193 | 135 |
| | Group 6 | 392 | 176 | 117 |
| coated | Group 7 | 412 | 206 | 111 |
| | Group 8 | 375 | 168 | 93 |
| | Group 9 | 335 | 144 | 91 |

Reduction in blood glucose was observed in Groups 4 to 9 where animals were administered with some form of costus extract. Group 7 to 9 showed the lowest blood sugar level after 30 days of administration. Extract coated with coating showed significantly better results, more than 50% reduction in blood glucose in 15 days and more than 70 percent reduction by day 30 were observed in coated groups (Group 7, 8 and 9).

Costus extract administered in group 6 showed significant improvements in lowering blood glucose, slightly lower than the coated groups. Extracts of group 4 to 6 were administered at a higher dosage than the coated once.

Oral glucose tolerance test (OGTT) was performed following a glucose challenge of 2 g/kg by oral gavages. It was to test the ability of body cells to absorb glucose after you ingest a given amount of sugar. Blood glucose was recorded at 30 min (PPG₁), 60 min (PPG₂), 120 min (PPG₃) and 180 min (PPG₄), after the glucose challenge on day 1, day 15 and day 30 of the study.

Postprandia glucose level (mg/dl) of rats in streptozotocin-induced diabetes Day 1. The results of the study are presented in Table. 4.

TABLE 4a
Day 1

| | PPG | PPG | PPG | PPG |
|---|---|---|---|---|
| Groups | 30 min | 60 min | 120 min | 180 min |
| Group 1 | 147 | 128 | 109 | 101 |
| Group 2 | 296 | 324 | 267 | 224 |
| Group 3 | 294 | 232 | 226 | 158 |
| Group 4 | 288 | 201.6 | 144 | 109.4 |
| Group 5 | 283 | 189.61 | 133.01 | 99 |
| Group 6 | 294 | 188.16 | 129.36 | 91 |
| Group 7 | 288 | 233.28 | 129.6 | 95 |
| Group 8 | 283 | 226.4 | 107.54 | 96.2 |
| Group 9 | 294 | 226.38 | 99.96 | 94.1 |

Post prandial glucose level (mg/dl) of rats in streptozotocin-induced diabetes Day 15.

TABLE 4b
Day 15

| | PPG | PPG | PPG | PPG |
|---|---|---|---|---|
| Groups | 30 min | 60 min | 120 min | 180 min |
| Group 1 | 146 | 131 | 112 | 108 |
| Group 2 | 301 | 333 | 275 | 230 |
| Group 3 | 285 | 225 | 219 | 153 |
| Group 4 | 273.6 | 191.52 | 136.8 | 103.9 |
| Group 5 | 268.85 | 180.13 | 126.3 | 94.1 |
| Group 6 | 279.3 | 178.7 | 122.9 | 86.6 |
| Group 7 | 273.6 | 221.6 | 123.12 | 90.3 |
| Group 8 | 268.85 | 215.08 | 102.1 | 91.4 |
| Group 9 | 279.3 | 215.0 | 94.9 | 89.4 |

Post prandial glucose level (mg/dl) of rats in streptozotocin-induced diabetes Day 30.

TABLE 4c
Day 30

| | PPG | PPG | PPG | PPG |
|---|---|---|---|---|
| Groups | 30 min | 60 min | 120 min | 180 min |
| Group 1 | 149 | 130 | 99 | 110 |
| Group 2 | 310 | 343 | 283 | 237 |
| Group 3 | 276 | 218 | 212 | 148 |
| Group 4 | 259.92 | 181.9 | 129.9 | 98.8 |
| Group 5 | 255.4 | 171.1 | 120.0 | 94.5 |
| Group 6 | 265.3 | 169.8 | 116.7 | 90.2 |
| Group 7 | 259.9 | 210.5 | 116.9 | 90.9 |
| Group 8 | 255.4 | 204.3 | 97.0 | 91.9 |
| Group 9 | 265.3 | 204.3 | 90.2 | 90.2 |

From group 4 to 9 there shows improvement in glucose tolerance test from day 1 to day 30. In Group 8 and 9 animals, the glucose level was brought to a normal level much faster in comparison to other groups.

Group 4 to 7 do manage to bring glucose level down close to normal for 180 min and there was steady improvement in each group from day 1 to day 30.

Among group 4 to 7, animals in group 7 had shown much better clinically significant improvement than the rest and at a dosage half of what was administered to animals in group 4 to 6.

Among all the groups from 4 to 9, group 8 and 9 animals have shown the most promising results. Among group 8 and 9 there was not much difference in final result, but the quantity of drug administered to animals in group 9 was one fifth of what was administered to animals in group 8 and group 7, and one tenth of what was administered to animals in group 4 to 6.

### Example 9

High Fat Diet-Induced Insulin Resistance Model Study to Assess the Effect of Various Costus Extracts is Shown.

Evaluating the effect with different samples of costus extraction high-fat diet-induced, insulin resistance model, with adult Wistar rats to study Type 2 diabetes was shown.

The study was designed to evaluate the effect of different fractions and compositions of costus on high fat-induced resistance Type-2 diabetic model (HFD). Adult Wistar rats were allowed free access to food and water. They were housed under identical standard conditions. The animals were kept for acclimatisation for a period of 7 days. After 7 days, the animals were randomly divided into six groups of six rats each. The normal control group was treated with normal regular diet and the rest of the animals were fed with high-fat diet for 6 weeks. After 6 weeks, Sample-1, Sample-3, and Product-2 were administered at a specific dosage as per their groups for 6 consecutive weeks. The experimental design was as follows:

Group 1—Normal control

Group 2—Diabetic control—untreated diabetic animals

Group 3—Positive control—diabetic animals administered with glibenclamide at 1 mg/kg Body wt orally for 30 days.

Group 4—diabetic animals administered with Sample-1 at 300 mg/kg Body wt orally for 30 days.

Group 5—diabetic animals administered with Sample-3 at 100 mg/kg Body wt orally for 30 days.

Group 6—diabetic animals administered with of Product-2 at 50 mg/kg Body wt orally for 30 days.

Observations

Body weights were determined weekly. At the end of experimental period, blood samples were collected to determine plasma glucose, insulin, glycosylated haemoglobin, triglycerides, and total cholesterol, LDL, HDL and VLDL.

### Body Weight

The results of the body weight of the animals are displayed in Table 5a. At the end of 6 weeks, the mean body weight of all the animals in each group increased about 260 g to nearly 300 g. After the end of 12 weeks, the sample treated groups showed a reduction in the body weight. Variations in the effect were observed among the treated groups. Among the sample treated groups with various costus extracts, promising results were observed in Group 4, Group 5 and Group 6.

In all the high-fat diet fed groups an increase of about 50 g body weight was observed, in the next 6 weeks a weight gain of about 48 g was observed in Group 2, where only high-fat diet was being fed. Group 4 to Group 6 where the animals were fed with different forms of costus extracts showed a dip in weight gain, in some cases the body weight of animals reached close to Group 1 animals.

Effect of various costus groups on body weight of the animals.

TABLE 5a

| | Pre-treatment | | Average Body weight | | | |
|---|---|---|---|---|---|---|
| | period body weight | | at weekly interval | | | |
| | (in grams) | | (in grams) | | | |
| Weeks | 0 | 2 | 3 | 4 | 5 | 6 |
| Gr. 1 | 221 | 226 | 226 | 224 | 225 | 223 |
| Gr. 2 | 217 | 260 | 282 | 292 | 303 | 308 |
| Gr. 3 | 216 | 259 | 274 | 280 | 270 | 248 |
| Gr. 4 | 210 | 252 | 273 | 281 | 268 | 260 |
| Gr. 5 | 212 | 254 | 265 | 277 | 256 | 239 |
| Gr. 6 | 214 | 256 | 256 | 267 | 250 | 235 |

### Blood Glucose

The results of the blood glucose level are depicted in Table. 5b. The results showed a reduction in the blood glucose level for all the sample treated groups. The highest reduction in blood glucose was observed in group 6 and Group 5.

TABLE 5b

| | Blood glucose |
|---|---|
| Groups | (mg/dl) |
| Group. 1. | 88 |
| Group. 2. | 495 |
| Group. 3. | 142 |
| Group. 4. | 165 |
| Group. 5. | 104 |
| Group. 6. | 91 |

### Lipid Profile

The results of the total cholesterol, triglycerides, LDL, HDL and VLDL are given in Table. 5c. The TC level of HFD treated group was high. All the sample treated groups showed a reduction in TC level. Similarly TG, LDL and VLDL showed higher values in HFD treated group. The LDL values of HFD treated group were low. But all the sample treated groups showed a reduction in the TG, LDL, VLDL and an increase in HDL. Among the sample treated groups, group 7 showed a reduction in the lipid profile which was almost similar to the standard drug (metformin) treated group.

Effect of costus on lipid profile in high fat diet induced diabetes mellitus.

TABLE 5c

| | TC | TG | LDL | HDL | VLDL |
|---|---|---|---|---|---|
| Groups | (mg/dl) | (mg/dl) | (mg/dl) | (mg/dl) | (mg/dl) |
| Group. 1. | 91.4 | 42 | 51 | 32 | 8.4 |
| Group. 2. | 118.2 | 66 | 73 | 32 | 13.2 |
| Group. 3. | 91.4 | 47 | 55 | 27 | 9.4 |
| Group. 4. | 96 | 45 | 58 | 29 | 9 |
| Group. 5. | 98.2 | 46 | 59 | 30 | 9.2 |
| Group. 6. | 92.2 | 41 | 53 | 31 | 8.2 |

### Serum Insulin Levels

The results of serum insulin and glycosylated haemoglobin are shown in Table. 5d. The HFD treated group showed an increase in insulin values. On the other hand, the sample treated groups (group 4, 5 & 6) showed a reduction in the serum insulin and glycosylated haemoglobin values. Among the samples treated group, the effect was more in group 5 which showed a more similar activity to the standard drug treated group.

Effect of costus on serum insulin, leptin and glycosylated haemoglobin levels in high-fat diet induced diabetes mellitus.

TABLE 5d

| | Insulin | Glycosylated Hb |
|---|---|---|
| | (ng/ml) | (% of total Hb) |
| Group. 1. | 0.7 | 0.5 |
| Group. 2. | 3.8 | 1.0 |
| Group. 3. | 1.7 | 0.5 |
| Group. 4. | 2.2 | 0.6 |
| Group. 5. | 1.7 | 0.5 |
| Group. 6. | 2.0 | 0.5 |

The above studies were carried out to evaluate the ability of different type of costus Composition for the treatment of Type-2 diabetes. Various samples of costus were tested for their ability to reduce glucose level in blood, modulate lipid profile, serum insulin and glycosylated haemoglobin levels.

It was observed that animals in test sample administered groups i.e. group 4 to group 6 showed positive results. Group 6 managed to restrain the rise in blood glucose level and the increase in body weight close to normal.

Group 5 animals with double the dose of group 6 showed significant restrain in the rise in blood glucose level and increase in body weight close to normal.

Group 6, group 5 and group 4 showed a significant reduction in total cholesterol even on a high fat diet. Group 6 animals showed slightly better results at a much lower dose, half the dose of group 5 and six times lower than group 4.

In light of the studies conducted, as illustrated in the example given above it can be said that coated costus composition such as Product-2 were better than their counter part because of their greater bioavailability and the constituents show greater bioactivity. It was also noted that the triterpenoids enriched extract also showed superior results over standard solvent extract.

### Example 10

Comparing the Activity of Coated Costus Extract with Costus Extract in Glucose Tolerance Test.

Adult Wistar rats weighing 200-250 gm were selected at random; the animals were kept in standard animal house conditions at 24±2° C., 65% relative humidity and 12 h light/dark cycle. The animals were acclimatized for a period of five days and fed with standard pellet diet and water. Six healthy rats were separated at random from the lot and diabetes was induced in rest by a single intra-peritoneal injection of streptozotocin (STZ) at 35 mg/kg.

Fasting blood sugar (FBS) was measured after seven days of STZ injection and rats having FBS more than 200 mg/dl were considered as diabetic. The diabetic rats were separated out and randomly divided into 4 groups having six rats in each. Six normal healthy rats were allocated to Group I; diabetic rats were randomly allocated to Groups 2 to 5.

For next thirty days the test drugs were administered to animals in each group from Group 3 to Group 5. Just 30 minutes before glucose challenge test a specific dose of test drug was administered to animals in each of the Group 3 to Group 5. Test drugs were selected from Sample 2 (from example 2), excipient and Product-2 (from example 4) and they were administered through oral gavages. The dosages for Sample-2 was 40 mg/Kg Body weight, dosage of excipient was 60 mg/Kg Body weight and dosages for Product-2 was 100 mg/kg Body weight. The excipient fed to the animals was Hicap, a starch, it was the same excipient used in Example 4 for the preparation of Product 2.

Group 1—Normal healthy animals as control.

Group 2—Diabetic animals administered with no drugs.

Group 3—Diabetic animals administered with excipient at 60 mg/Kg Body weight twice daily.

Group 4—Diabetic animals administered with Sample-2 at 40 mg/Kg Body weight twice daily.

Group 5—Diabetic animals administered with Product-2 at 100 mg/Kg Body twice daily.

The animals were tested for glucose tolerance as per below experimental design:

Fasting blood glucose was tested just before each OGTT was performed. Fasting blood glucose test (mg/dl) results are provided in table 6.

TABLE 6

| | FSB (mg/dl) | | |
|---|---|---|---|
| | 1^{st} | 15^{th} | 30^{th} |
| | Day | Day | Day |
| Group 1: Control group | 94 | 94 | 96 |
| Group 2: Diabetic animals | 215 | 218 | 221 |
| administered with no drugs. | | | |
| Group 3: Diabetic animals | 214 | 219 | 220 |
| administered with excipient | | | |
| at 60 mg/Kg Body weight. | | | |
| Group 4: Diabetic animals | 216 | 203 | 169 |
| administered with Sample-2 | | | |
| at 40 mg/kg Body weight. | | | |
| Group 5: Diabetic animals | 218 | 123 | 98 |
| administered with Product-2 | | | |
| at 100 mg/kg Body weight. | | | |

Oral glucose tolerance test (OGTT) was performed following a glucose challenge of 2 g/kg by oral gavages. It is to test the ability of body cells to absorb glucose after you ingest a given amount of sugar. Blood glucose was recorded at 30 min, 60 min, 120 min and 180 min, after the glucose challenge on day of the study.

Postprandial glucose level (mg/dl) of rats in streptozotocin-induced diabetics.

TABLE 7a
1^{st} day of test

| | glucose level (mg/dl) | | | | |
|---|---|---|---|---|---|
| | Zero | 30 | 60 | 120 | 180 |
| | min | min | min | min | min |
| Group 1: Control group | 94 | 149 | 142 | 127 | 98 |
| Group 2: Diabetic animals | 215 | 297 | 321 | 287 | 268 |
| administered with no drugs. | | | | | |
| Group 3: Diabetic animals | 214 | 299 | 324 | 288 | 270 |
| administered with excipient | | | | | |
| at 60 mg/Kg Body weight. | | | | | |
| Group 4: Diabetic animals | 216 | 295 | 268 | 205 | 186 |
| administered with Sample-2 | | | | | |
| at 40 mg/kg Body weight. | | | | | |
| Group 5: Diabetic animals | 218 | 288 | 240 | 114 | 105 |
| administered with Product-2 | | | | | |
| at 100 mg/kg Body weight. | | | | | |

TABLE 7b
15^{th} Day of the test

| | glucose level (mg/dl) | | | | |
|---|---|---|---|---|---|
| | Zero | 30 | 60 | 120 | 180 |
| | min | min | min | min | min |
| Group 1: Control group | 94 | 147 | 145 | 128 | 96 |
| Group 2: Diabetic animals | 218 | 299 | 330 | 290 | 278 |
| administered with no drugs. | | | | | |
| Group 3: Diabetic animals | 219 | 301 | 332 | 291 | 280 |
| administered with excipient | | | | | |
| at 60 mg/Kg Body weight. | | | | | |
| Group 4: Diabetic animals | 203 | 240 | 265 | 206 | 179 |
| administered with Sample-2 | | | | | |
| at 40 mg/kg Body weight. | | | | | |
| Group 5: Diabetic animals | 129 | 193 | 151 | 130 | 97 |
| administered with Product-2 | | | | | |
| at 100 mg/kg Body weight. | | | | | |

TABLE 7c
30^{th} day of the test

| | glucose level (mg/dl) | | | | |
|---|---|---|---|---|---|
| | Zero | 30 | 60 | 120 | 180 |
| | min | min | min | min | min |
| Group 1: Control group | 96 | 150 | 140 | 125 | 95 |
| Group 2: Diabetic animals | 221 | 301 | 338 | 291 | 283 |
| administered with no drugs. | | | | | |
| Group 3: Diabetic animals | 220 | 302 | 339 | 294 | 285 |
| administered with excipient | | | | | |
| at 60 mg/Kg Body weight. | | | | | |
| Group 4: Diabetic animals | 169 | 215 | 236 | 185 | 153 |
| administered with Sample-2 | | | | | |
| at 40 mg/kg Body weight. | | | | | |
| Group 5: Diabetic animals | 98 | 157 | 134 | 121 | 94 |
| administered with Product-2 | | | | | |
| at 100 mg/kg Body weight. | | | | | |

It was observed that by the 30^{th} day of the study Fasting blood glucose level in the Group 5 animals had reached at par with the normal value or the glucose value of animals from Group 1 (normal control).

The average Fasting blood glucose level has fallen from 218 mg/dl on day one to 98 mg/dl in day 30 for the animals in group 5, that is a 120 mg/dl reduction in blood glucose. At the same time the fasting blood glucose reduction observed in Group 4 after 30 days was only 47 mg/dl, the blood glucose reduction in Group 3 animals was 2.5 times less than Group 5.

On day one of the glucose tolerance test not much improvement was seed in most of the groups, the blood glucose level rose to about 300 mg/dl in 60 minutes in diabetes induced animals and came down to about 270-250 mg/dl in 180 minutes. In Group 5 a significant reduction in blood sugar was observed on day one. In Group 5 the blood sugar was brought down to below 110 mg/dl with 180 minutes.

By the 15^{th} day significant improvement was observed in fasting blood sugar level for Group 5 animals. The blood glucose level did not pass 200 mg/dl and was brought down to 116 mg/dl by 180^{th} minute. The Group 4 animals showed improvement compared to Group 2 and Group 3, also managed to bring the blood glucose level below 200 mg/dl by 180^{th} minute but it was still 59 mg/dl short of Group 5.

By the 30^{th} day the reading of Group 1 animals and Group 5 animals were almost identical, as if the group 5 animals were not diabetic any more. On the contrary the average blood glucose could come down to only 153 mg/dl after 180 minutes of glucose challenge test in animals from Group 4, though it was better than the reading observed in Group 2 and Group 3 but it was 54 mg/dl short of group 4 readings.

The active part, costus extract, in the test drug administered to animals in group 4 and group 5 were same. 40 mg/Kg body weight of costus extract was administered twice in both the groups. In group 5 the drug was blended with excipient and provided with an enteric coating. It was observed that even though the active drug administered to animals in group 4 and Group 5 was same a significant improvement was observed in animals administered with costus extract (sample 2) with the coating. It can be concluded that the coating has improved the bioactivity of the sample 2 (costus extract).

### Example 11

Comparing the Activity of Coated Costus Extract with Costus Extract for Type 2 Diabetics.

Wistar rats were selected at random; the animals were kept in standard animal house conditions at 24±2° C., 65% relative humidity and 12 h light/dark cycle. The animals were acclimatized for a period of five days and fed with standard pellet diet and water. Fasted male Wistar rats were intraperitoneally injected with freshly prepared solution of streptozotocin (STZ) (35 mg/kg). After injection, all the animals were continued on the standard rodent diet for 2 weeks. At the end of 2 weeks after STZ injection; 1 ml of blood was collected from retro-orbital plexus of each rat. Blood was allowed to clot for 30 min the serum was separated by centrifuging at 1008 G-force for 20 min at 8.0° C. in a cold centrifuge. The serum separated from the blood of each animal was used for the estimation of glucose. All the samples were analysed calorimetrically in Autoanalyzer (MERCK MICROLAB 300) using commercial kits. The development of hyperglycaemia in these rats was thus confirmed by measuring fasting blood glucose levels. The rats with the fasting PGL of ≥200 mg dl−1 were considered diabetic and selected for further dietary manipulation. 350 g of standard rodent diet was powdered and mixed with 60 g of lard, 45 g of yolk powder, and 45 g of plantation white sugar (HFD diet). The above mixture is pelleted and used to feed the rats for 2 weeks to induce diabetic dyslipidaemia. Required amount of the diet was freshly prepared on a daily basis. At the end of 2nd week the blood was collected from each animal for determination of glucose levels as described above.

After the second fasting blood glucose estimation, at the end of 4^{th} week, the rats were divided into 6 groups of 6 rats each: Group-1 consisted of age-matched non-diabetic normal rats that neither received streptozotocin nor the high-fat diet and served as normal; Group 2 consisted of diabetic rats without any drug treatment and served as untreated control; Group 3 consisted of diabetic rats and received standard drug, Glibenclamide. Group 4 consisted of diabetic rats and received excipients. Group 5 consisted of diabetic rats and sample 2. Group 6 consisted of diabetic rats and product 2. The high fat diet was continued for the rest of the study duration.

Study to compare the effect of Costus extract coated and uncoated.

Group1—Normal control treated with normal regular diet.

Group 2—Diabetic, HFD administered group with no medicine.

Group 3—Diabetic, HFD administered group treated with Glibenclamide at 2.5 mg/Kg p.o. body weight.

Group 4—Diabetic, HFD administered group treated with excipients at 60 mg/kg twice daily.

Group 5—Diabetic, HFD administered group treated with sample 2 for at 40 mg/kg twice daily.

Group 6—Diabetic, HFD administered group treated with product-2 at 100 mg/kg twice daily.

Body weights were determined biweekly. At the end of experimental period, blood samples were collected to determine plasma glucose, insulin, leptin, glycosylated hemoglobin, triglycerides, total cholesterol, LDL, HDL and VLDL.

Body Weight

Mean body weight and change in body weight during treatment. The results of the body weight of the animals are displayed in Table 8.

TABLE 8

| | Body weight at biweekly interval (in grams) | | | | |
|---|---|---|---|---|---|
| Groups | 0 | 2 | 4 | 6 | 8 |
| Group 1-Normal control, fed regular diet. | 162 | 165 | 168 | 170 | 173 |
| Neither receive streptozotocin nor the | | | | | |
| high-fat diet | | | | | |
| Group 2-Diabetic, HFD administered | 169.33 | 178.5 | 184.83 | 190.33 | 197 |
| group with no medicine. | | | | | |
| Group 3-Diabetic, HFD administered | 168 | 174 | 180 | 187 | 192 |
| group treated with Glibenclamide at 2.5 | | | | | |
| mg/Kg body weight. | | | | | |
| Group 4-Diabetic, HFD administered group | 168 | 176 | 185 | 192 | 196 |
| treated with excipients at 60 mg/kg. | | | | | |
| Group 5-Diabetic, HFD administered | 167 | 172 | 179 | 186 | 191 |
| group treated with sample 2 at 40 mg/kg. | | | | | |
| Group 6-Diabetic, HFD administered | 169 | 172 | 175 | 182 | 185 |
| group treated with product-2 at 100 mg/kg | | | | | |

It was observed that normal animals with no diabetes and did not receive HFD of Group 1 have gained about 11 gm. At the same time, diabetic animals fed with HFD and no medicine has gained about 28 gm in 28 days. That is about double the increase in body weight. Group 3 animals treated with Glibenclamide showed increase in body weight but slightly less than Group 2 animals.

Group 5 and Group 6 animals showed improvement in controlling the body weight, especially Group 6 animals. After 28 days of study the average body weight in Group 5 animals have increased by 24 gm, at the same time the increase in average body weight of animals in Group 6 is just 16 gm.

Even though the active test drug in both the Groups 5 and 6 is the same, 40 mg/Kg body weight, but still the activity of drug provided in Group 6 (product 2) is superior to the drug given in Group 5 (sample 2).

TABLE 9

| | TC | TG | LDL | HDL | VLDL |
|---|---|---|---|---|---|
| Groups | (mg/dl) | (mg/dl) | (mg/dl) | (mg/dl) | (mg/dl) |
| Group 1-Normal control, fed regular diet. | 120.2 | 86 | 76 | 27 | 17.2 |
| Neither receive streptozotocin nor the high-fat | | | | | |
| diet | | | | | |
| Group 2-Diabetic, HFD administered group | 187.8 | 179 | 133 | 19 | 35.8 |
| with no medicine. | | | | | |
| Group 3-Diabetic, HFD administered group | 136.4 | 97 | 92 | 25 | 19.4 |
| treated with Glibenclamide at 2.5 mg/Kg | | | | | |
| body weight. | | | | | |
| Group 4-Diabetic, HFD administered group | 189.4 | 177 | 136 | 18 | 35.4 |
| treated with excipients at 60 mg/kg. | | | | | |
| Group 5-Diabetic, HFD administered group | 163.6 | 128 | 110 | 28 | 25.6 |
| treated with sample 2 at 40 mg/kg. | | | | | |
| Group 6-Diabetic, HFD administered group | 131.2 | 91 | 80 | 33 | 18.2 |
| treated with product-2 at 100 mg/kg | | | | | |

After 28 days of the test drug study the lipid profile was tested and the following observations were made;

Product 2 is the most active test sample with significant improvement in reducing total cholesterol, triglyceride, LDL and VLDL.

Compared to Group 5 animals about double the total cholesterol reduction were observed in Group 6 animals.

The reduction in triglyceride, VLDL and LDL was also significant in Group 6 animals compared to group 5 animals.

Even though the active test drug in both the Groups 5 and 6 is the same, 40 mg/Kg body weight, but still the activity of drug provided in Group 6 (product 2) is superior to the drug given in Group 5 (sample 2).

Serum Insulin Levels

The results of serum insulin and glycosylated hemoglobin after 28 days for treatment are shown in Table 10.

TABLE 10

| | | Glycosylated |
|---|---|---|
| | Insulin | Hb (% of |
| Groups | (ng/ml) | total Hb) |
| Group 1-Normal control, fed regular | 1.58 | 0.5 |
| diet. Neither receive streptozotocin | | |
| nor the high-fat diet | | |
| Group 2-Diabetic, HFD administered | 1.11 | 1 |
| group with no medicine. | | |
| Group 3-Diabetic, HFD administered | 1.58 | 0.5 |
| group treated with Glibenclamide at | | |
| 2.5 mg/Kg body weight. | | |
| Group 4-Diabetic, HFD administered | 1.11 | 1.1 |
| group treated with excipients at 60 | | |
| mg/kg. | | |
| Group 5-Diabetic, HFD administered | 1.19 | 0.9 |
| group treated with sample 2 at 40 | | |
| mg/kg. | | |
| Group 6-Diabetic, HFD administered | 1.69 | 0.5 |
| group treated with product-2 at 100 | | |
| mg/kg | | |

It was observed that the insulin production has significantly increased during the test period, especially for Group 6 animals treated with Product-2. The improvement observed in Group 6 is significantly better than Group 5 animals.

What is claimed is:

1. An enteric coated composition comprising a core and an enteric coating layer, wherein the core comprises a Costus pictus constituent and a pharmaceutical carrier, and, wherein the Costus pictus constituent and the pharmaceutical carrier are blended in a ratio of 10:1 to 1:10.

2. The enteric coated composition of claim 1, wherein the Costus pictus constituent are selected from the group consisting of Costus pictus plant juice, concentrated Costus pictus plant juice, powder of dried leaf of Costus pictus, solvent extract of Costus pictus, polyphenol enriched extract of Costus pictus, protein enriched extract of Costus pictus, water insoluble triterpenoid enriched extract of Costus pictus and combinations thereof.

3. The enteric coated composition of claim 2, wherein the Costus pictus constituent is water insoluble triterpenoid enriched extract of Costus pictus leaf with enriched triterpenoids.

4. The enteric coated composition of claim 3, wherein the Costus pictus composition comprises by weight:
about 10% to about 95% triterpenoids; and,
about 0.1 to about 1% oxalic acid.

5. The enteric coated composition of claim 3, wherein the triterpenoids comprises by weight about 15% to 80%.

6. The enteric coated composition of claim 3, wherein the triterpenoids comprises by weight about 35% to 50%.

7. The enteric coated composition of claim 3, wherein the triterpenoids comprises by weight about 80% to 90%.

8. The enteric coated composition of claim 1, wherein the pharmaceutical carrier is selected from the group consisting of polyvinyl-pyrrolidone, cellulose derivatives including hydroxypropyl methylcellulose and hydroxypropyl cellulose, cyclodextrins, gelatines, hypromellose phthalate, sugars and polyhydric alcohols, Calcium Sulphate Dihydrate, Dibasic Calcium Phosphate, Sorbitol, Mannitol, Anhydrous Lactose, Dextrose, gums, Gum Acacia, Gum Tragacanth, Ethylcellulose, Methylcellulose, Polyvinyl alcohol, sodium carboxy methyl cellulose, silicon dioxide, polyethylene glycol 400, Polyethylene glycol 4000, starch, porous silica carriers, aluminum silicate, calcium silicate, sugars, magnesium stearate, cellulose, calcium phosphate and combinations thereof.

9. The enteric coated composition of claim 1, wherein the enteric coating material is selected from the group consisting of combination of aqueous ethyl cellulose and sodium alginate, poly (methacrylic acid-co-methyl methacrylate), cellulose acetate phthalate, cellulose acetate trimellitate, hydroxypropyl methylcellulose phthalate, hydroxypropyl methylcellulose acetate succinate, and combinations thereof.

10. The enteric coated composition of claim 9, wherein the enteric coating material is a combination of aqueous ethyl cellulose and sodium alginate.

11. The enteric coated composition of claim 9, wherein the enteric coating layer further comprises an excipient selected from the group consisting of ethyl cellulose, sodium alginate, esters of aleurtic acid poly (vinyl acetate phthalate), hydroxypropyl methylcellulose, acetaldehyde dimethyl cellulose acetate, shellac, chitosan, fatty acids, zein, waxes, plant fibers, modified starch, aluminum silicate, calcium silicate, sugars, magnesium stearate, cellulose and calcium phosphate, polysorbate, surfactants and combinations thereof.

12. The enteric coated composition of claim 8, wherein the pharmaceutical carrier is a modified starch

13. The enteric coated composition of claim 1, wherein the core is in a liquisolid form.

14. An oral dosage form of the enteric coated composition of claim 1, wherein the oral dosage form is selected from the group consisting of tablet, mini tablet, micro encapsulate, pill, capsule, soft gel capsule, and, hard gel capsule.

15. A method of enhancing antidiabetic activity comprising administering the enteric coated extract of claim 1, wherein antidiabetic activity is selected from the group consisting of controlling level of glucose in blood, causing regeneration of β-cells, preventing production of glucose in the liver, lowering hyperglycemia, increasing insulin secretion in Type1 diabetic patient, treating Type 2 diabetes by decreasing HbAlc level, balancing lipid profiles, increasing liver glycogen, and increasing muscle glycogen.

16. A method to improve yield of an extract of Costus pictus during manufacturing, the method to improve yield comprising combining an extract prepared from a juice of Costus pictus with an extract prepared from a pulp of Costus pictus, wherein the method to improve yield comprises:
a) crushing plant parts of Costus pictus to extrude the juice of Costus pictus and the pulp of Costus pictus,
b) separating the juice of Costus pictus from the pulp of Costus pictus;
c) extracting the juice of Costus pictus from step b) by a method comprising:
c1) treating the juice of Costus pictus with ethyl acetate to obtain a liquid phase ethyl acetate extract of juice of Costus pictus;
c2) washing the liquid phase ethyl acetate extract of juice of Costus pictus of step c1) with deionized water in presence of NaCl and collecting a first ethyl acetate phase, wherein the first ethyl acetate phase is a first water insoluble triterpenoid enriched ethyl acetate extract;
c3) concentrating the first water insoluble triterpenoid enriched ethyl acetate extract from step c2) to obtain a concentrate of the first water insoluble triterpenoid enriched ethyl acetate extract of the juice of Costus pictus;
d) extracting the pulp of Costus pictus of step b) by a method comprising:
d1) extracting the pulp of Costus pictus extract with ethyl acetate to obtain a first liquid phase ethyl acetate extract of pulp of Costus pictus;
d2) washing the liquid phase ethyl acetate extract of pulp of Costus pictus of step d1) with deionized water and NaCl and collecting a second ethyl acetate phase, wherein the second ethyl acetate phase is a second water insoluble triterpenoid enriched ethyl acetate extract of the pulp of Costus pictus;
d3) concentrating the second water insoluble triterpenoid enriched ethyl acetate extract of the pulp of Costus pictus from step d2) to obtain a concentrate of the second water insoluble triterpenoid enriched ethyl acetate extract of the pulp of Costus pictus;
e) combining the concentrate of the first water insoluble triterpenoid enriched ethyl acetate extract of juice of Costus pictus of step c3) with the concentrate of the second water insoluble triterpenoid enriched ethyl acetate extract of the pulp of Costus pictus of step d3) to obtain a third water insoluble triterpenoid enriched extract of Costus pictus, wherein separately extracting then combining extracts prepared from the juice of Costus pictus and the pulp of Costus pictus results in an improvement in yield of extract from Costus pictus.

17. A method of enriching triterpenoids in the improved yield extract of Costus pictus of claim 16, the method comprising:
g) dissolving the improved yield extract of Costus pictus of step e) of claim 16 in methanol and separating a solvent phase and a residue;
h) loading a wet packed column having HP20 in methanol with the solvent phase from step g);
i) eluting the product of step h) with ethyl acetate to obtain an ethyl acetate eluate; and,
j) concentrating the ethyl acetate eluate of step j) to obtain a triterpenoid enriched extract of Costus pictus, wherein the triperpenoid enriched extract of Costus pictus comprises about 80% to about 90% w/w of triterpenoids.

18. A method of preparing an enteric coated composition comprising a core and an enteric coating layer, wherein the core comprises a Costus pictus constituent and a pharmaceutical carrier, and, the enteric coating layer comprises an enteric coating material,
the method comprising:
mixing a Costus pictus constituent and pharmaceutical carrier to obtain a first uniform blend;
spray drying the uniform blend to obtain a powder;
fluidizing the powder;
spraying a coating solution onto the fluidized powder to obtain granules of the enteric coated composition comprising a core and an enteric coating layer, wherein the core comprises the Costus pictus constituent and the pharmaceutical carrier, and, wherein the Costus pictus constituent and the pharmaceutical carrier are blended in a ratio of 10:1 to 1:10.
